(19) United States
(12) Patent Application Publication
(10) Pub. No.: US 2014/0287933 A1
(43) Pub. Date: Sep. 25, 2014

(54) METHODS FOR MEASURING FEL d 1 CAT ALLERGEN

(71) Applicant: Savannah Tobin, Salem, OR (US)

(72) Inventor: Savannah Tobin, Salem, OR (US)

(21) Appl. No.: 14/197,452

(22) Filed: Mar. 5, 2014

Related U.S. Application Data
(60) Provisional application No. 61/782,402, filed on Mar. 14, 2013.

(51) Int. Cl. C12Q 1/68

(52) U.S. Cl. CPC C12Q 1/6888
USPC 506/2; 435/6.18; 435/6.1; 435/6.12; 506/9; 435/6.11

(57) ABSTRACT

The present invention relates to a noninvasive method for quantifying Fel d 1 cat allergen expression levels—the major allergen responsible for cat allergies—in individual cats (Felis catus).

### CROSS REFERENCE TO RELATED APPLICATIONS

This application claims the benefit of U.S. Provisional Patent Application Ser. No. 61/782,402, filed Mar. 14, 2013 and is incorporated herein by reference.

### FIELD OF THE INVENTION

This invention relates to quantifying Fel d 1 cat allergen n expression levels—the major allergen responsible for cat allergies—in individual cats (Felis catus).

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Over 600 million cats (Felis catus) live in the company of humans worldwide. Although the domestic cat has lived among humans since 7500 B.C., many people are allergic to the tiny protein cats secrete in their salivary and sebaceous glands. This protein, Felis domesticus 1 (Fel d 1), causes asthmatics to react to cats and approximately 15% of the world's population is deemed allergic to cats. It is a common misconception that cat fur is responsible for inducing allergic response. The protein Fel d 1 originates in cat saliva, and as cats groom themselves they coat their fur in the allergenic protein. Despite the high number of people who suffer from cat allergies, one third of the affected population is believed to keep cats as pets regardless of their allergic condition. Although the purpose of Fel d 1 within the cat's body is currently unknown, it is clear that the protein is the major allergen responsible for cat allergies, and although all cats produce Fel d 1, specific cat breeds such as the Russian Blue and Siberian have been known to produce significantly lower levels of the allergenic protein and are therefore anecdotally hypoallergenic. While little scientific evidence backs the claim of hypoallergenic cats, people with allergies have noticed lower allergic reactivity when exposed to them. As each individual cat produces its own unique level of Fel d 1, it is plausible that certain cats might cause allergic reactions more infrequently and with less severity. A significant portion of the global population suffers the effects of cat allergies; however, if a methodology were developed to quantify Fel d 1, cats identified as being less likely to cause allergic reactions could be adopted by people sensitive to Fel d 1. Additionally, the validity of hypoallergenic cats could be tested. There is a need in the art for easy and accurate quantification of Fel d 1. The present invention satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

This invention relates to non-invasively quantifying Fel d 1 cat allergen n expression levels in individual cats (Felis catus), by quantifying mRNA from cat saliva. The methods do not require using a salivant to increase saliva production, but instead can be used on the saliva that cats produce naturally.

Thus, in some embodiments, the present invention provides a method for determining levels of Fel d 1 proteins in a saliva sample obtained from a cat (Felis catus), comprising the steps of obtaining the saliva sample from the cat; isolating mRNA from the saliva sample; and quantifying the mRNA coding for Fel d 1 in the saliva sample.

In certain aspects of this embodiment, the method further comprises quantifying mRNA coding for at least one housekeeping gene, and in some aspects, the housekeeping gene is one or both of glyceraldehyde 3-phosphate dehydrogenase (GAPDH) or ribosomal protein S7 (RPS7).

In some aspects, the methods of the invention further comprise, after the isolating step and before the quantifying step, reverse transcribing the mRNA coding for Fel d 1 and the at least one housekeeping gene to produce cDNAs, and performing qPCR or dPCR on the cDNA products. In some aspects, the primers for Fel d 1 comprise at least one of the following pairs of primers: AAGGATGTTAGACGCAGCCC and CAACATCCCTCTTCACGGCT or TTGCTACGTGGAGAACGGAC and TTGCTGGAGCTGATGGTTGT.

In some aspects, as an alternative to quantitative PCR or digital PCR, cDNAs produced by reverse transcription are quantified by performing massively parallel sequencing.

In some aspects of this embodiment of the invention, the amount of sample per reaction is less than 10 μl, in preferred aspects, the amount of sample per reaction is less than 4 μl.

In yet another embodiment, the present invention provides a method for determining levels of Fel d 1 proteins in a saliva sample obtained from a cat (Felis catus), comprising the steps of obtaining the saliva sample from the cat; reverse transcribing the mRNA coding for Fel d 1 and at least one housekeeping gene; performing qPCR, dPCR or massively parallel sequencing on the cDNA products; and quantifying the mRNA coding for Fel d 1 in the sample.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The terms “complementary” or “complementarity” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100% complementarity, and even more preferably with 100% complementarity. Alternatively, substantial complementarity exists when an RNA or DNA strand hybridizes under selective hybridization conditions to its complement. Selective hybridization conditions include, but are not limited to, stringent hybridization conditions. Stringent hybridization conditions will typically include salt concentrations of less than about 1 M, more usually less than about 500 mM and preferably less than about 200 mM. Hybridization temperatures are generally at least about 2° C. to about 6° C. lower than melting temperatures (Tₘ).

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “melting temperature” or Tm is commonly defined as the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+16.6(log 10[Na+])0.41(%[G+C])−675/n−1.0m, when a nucleic acid is in aqueous solution having cation concentrations of 0.5 M or less, the (G+C) content is between 30% and 70%, n is the number of bases, and m is the percentage of base pair mismatches (see, e.g., Sambrook J et al., Molecular Cloning, A Laboratory Manual, 3rd Ed., Cold Spring Harbor Laboratory Press (2001)). Other references include more sophisticated computations, which take structural as well as sequence characteristics into account for the calculation of Tₘ.

As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [S]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP.

The term “oligonucleotide” or “oligo” as used herein refers to a linear oligomer of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more. Suitable nucleic acid molecules may be prepared by the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both incorporated herein by reference, or by other chemical methods such as using a commercial automated oligonucleotide synthesizer.

As used herein the term “polymerase” refers to an enzyme that links individual nucleotides together into a long strand, using another strand as a template. There are two general types of polymerase-DNA polymerases, which synthesize DNA, and RNA polymerases, which synthesize RNA. Within these two classes, there are numerous sub-types of polymerases, depending on what type of nucleic acid can function as template and what type of nucleic acid is formed.

As used herein “polymerase chain reaction” or “PCR” refers to a technique for replicating a specific piece of target DNA in vitro, even in the presence of excess non-specific DNA. Primers are added to the target DNA, where the primers initiate the copying of the target DNA using nucleotides and, typically, Taq polymerase or the like. By cycling the temperature, the target DNA is repetitively denatured and copied. A single copy of the target DNA, even if mixed in with other, random DNA, can be amplified to obtain billions of replicates. The polymerase chain reaction can be used to detect and measure very small amounts of DNA and to create customized pieces of DNA. In some instances, linear amplification methods may be used as an alternative to PCR.

Generally, a “primer” is an oligonucleotide used to, e.g., prime DNA extension, ligation and/or synthesis, such as in the synthesis step of the polymerase chain reaction or in the primer extension techniques used in certain sequencing reactions or in reverse transcription.

“Reverse transcriptase” refers to an enzyme used to generate complementary DNA (cDNA) from an RNA, typically mRNA, template.

The term “sequencing” as used herein refers generally to any and all biochemical methods that may be used to determine the order of nucleotide bases including but not limited to adenine, guanine, cytosine and thymine, in one or more molecules of DNA. As used herein the term “sequence determination” means using any method of sequencing known in the art to determine the sequence nucleotide bases in a nucleic acid.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; and Berg et al. (2002) Biochemistry, 5^{th} Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

The present invention is drawn to measuring the level of expression of Fel d 1 from cat saliva. The methods of the invention are noninvasive and allow for accurate measurement of Fel d 1 expression by measuring mRNA levels, most preferably through reverse transcription of the mRNA into cDNA, then quantifying the cDNAs using qPCR, dPCR or next generation (massively parallel) sequencing. To date, an ELISA assay has been developed to measure Fel d 1 protein directly; however, the ELISA assay requires administration of a salivant to the cat to produce the large amount of saliva needed to run the assay. The methods of the present invention require a greatly reduced amount of saliva—as much as 100-fold less or more—to measure Fel d 1 expression.

Sample collection for the present invention is straightforward. Sterile cotton swabs can be used, or, alternatively, saliva collection kits known in the art can be used, where such devices typically comprise a sterile absorbent device or swab or “sponge”, and a sterile container in which the device can be placed once it has been used and before sample processing. The methods of the present invention typically are used without first administering a salivant to the cat—instead, the methods of the present invention utilize collection of saliva that is naturally produced by the cat. However, because mRNA is used to quantify the expression of the Felis domesticus 1 (Fel d 1) protein, extreme care needs to be taken to assure that Ribonucleases (RNases) are avoided. Though studies show that saliva has natural stabilizing enzymes that keep mRNA intact for up to three months at room temperature (Wong, et al, Clinical Chemistry, 57(9):1295-302 (2011)), RNases are very stable and active enzymes that generally do not require cofactors to function, making RNases difficult to inactivate. Because even minute amounts are sufficient to destroy RNA, plasticware or glassware should never be used without first eliminating possible RNase contamination. That is, great care should be taken to avoid inadvertently introducing RNases into the RNA sample during or after the isolation procedure. In order to create and maintain an RNase-free environment, precautions should be taken. For example, use of sterile, disposable polypropylene tubes is recommended, and glassware, e.g., should be cleaned with a detergent, thoroughly rinsed, and oven baked at 240° C. for four or more hours. Additionally, gloves, a lab coat, sterile face mask and goggles should be worn, and preferably, all prep work should be performed in an area that has been scrubbed down and preferably is dedicated to working with RNA. The present methods are drawn to collection of saliva and are noninvasive; however, alternative methods may employ sample collection from the sebaceous glands of the cat.

mRNA can be isolated by any one of many methods known in the art. For example, Poly(A)-RNA preparation can be accomplished using cellulose-bound oligo-dT, but several other reagents have been developed, e.g., streptavidin-coupled magnetic beads used in combination with biotinylated oligo-dT or oligo-dT-coupled polystyrene-latex beads. The oligo-dT/carrier combinations are available separately from several manufacturers; however, one may find it more convenient to use a kit which has the advantage of containing most of the necessary reagents pre-packaged in RNase-free quality, e.g., POLYATTRACT® from Promega, POLYA SPIN™ from New England Biolabs or OLIGOTEX™ mRNA kit from Qiagen. Alternatively, one can simply treat the sample with RNase-free DNase to eliminate the DNA in the sample, thereby enriching the sample for RNA.

RT-PCR (reverse transcription PCR) is used to clone expressed genes by reverse transcribing mRNA into its DNA complement through the use of the enzyme reverse transcriptase. Subsequently, the newly synthesized cDNA is quantified using qPCR, dPCR or next generation (aka massively parallel) sequencing. The quantification of mRNA using RT-PCR can be achieved as either a one-step or a two-step reaction. The difference between the two approaches lies in the number of tubes used when performing the reverse transcription step and the subsequent PCR amplification step. In the one-step approach, the entire reaction from cDNA synthesis to qPCR or dPCR amplification occurs in a single tube. In contrast, the two-step reaction requires that the reverse transcriptase reaction and qPCR or dPCR amplification be performed in separate tubes. The one-step approach is thought to minimize experimental variation by containing all of the enzymatic reactions in a single environment.

One method for quantifying the cDNA resulting from the reverse transcription procedure in a sample is use of quantitative PCR or qPCR. qPCR follows the general principle of the polymerase chain reaction; the key feature of qPCR being that amplified DNA is detected as the reaction progresses in real time as opposed to standard PCR, where amplified DNA is detected only after the final reaction cycle. Two common methods for detection of products in real-time PCR are the use of non-specific fluorescent dyes that intercalate with any double-stranded DNA, and the use of sequence-specific DNA primers consisting of oligonucleotides that are labeled with a fluorescent reporter that permits detection only after hybridization of the primer with its complementary DNA target. qPCR typically is run in a real-time PCR instrument, where after each cycle levels of fluorescence are measured with a detector. The detection or reporter dye fluoresces only when bound to double-stranded DNA, that is, the PCR product and can be detected only when a threshold of PCR product has been produced. The earlier the cycle in which PCR product is detected, the more cDNA—hence mRNA—there is in the sample. A low CT value correlates with detectable PCR product at an early cycle, and a large CT value correlates with detectible PCR products at a later cycle. The concentration of the qPCR product can then be determined, e.g., with reference to a standard dilution or concentration can be relative to other samples.

An alternative method for quantifying cDNA in a sample is digital PCR or dPCR. With dPCR, the cDNA sample is partitioned so that individual nucleic acid molecules within the sample are localized in separate small volumes, such as in micro-well plates, capillaries, a phase emulsion, or in arrays of very small-volume chambers, typically in a dilution of only one molecule in every two chambers. As a result, each small volume will contain one or no molecules, resulting in a positive or negative PCR reaction, respectively. The separation of the nucleic acids allows for counting of individual molecules, resulting in a more reliable and sensitive measurement of nucleic acids than can be obtained by standard PCR or by qPCR, as amplification bias is effectively eliminated. After amplification, the nucleic acids are quantified by counting the number of locations that contain a PCR end-product; for example, by using differently-labeled oligonucleotide probes (see, e.g., Vogelstein and Kinzler, PNAS USA, 96:9236-41 (1999)).

A third method for quantifying the cDNA in a sample is next generation sequencing (NGS), also known as massively parallel sequencing (MPS), which also allows for single molecule counting, and thus increased accuracy. Current NGS methods and systems that allow for single molecule counting include pyrosequencing, as commercialized by 454 Life Sciences; sequencing by ligation, as commercialized in the SOLiD™ technology, by Life Technology, Inc., Carlsbad, Calif.; sequencing-by-synthesis methods using modified nucleotides, as commercialized in TRUSEQ™ and HISEQ™ technology by Illumina, Inc., San Diego, Calif.; PacBio RS by Pacific Biosciences of California, Inc., Menlo Park, Calif.; sequencing by ion detection technologies, as commercialized by Ion Torrent, Inc., South San Francisco, Calif.; and sequencing of DNA nanoballs, commercialized by Complete Genomics, Inc., Mountain View, Calif. It should be noted that many techniques for sample collection, sample processing, mRNA isolation or enrichment and nucleic acid quantification are known in the art, and the present invention should not be limited by the exemplary methods mentioned above, or in the Examples, below.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Saliva Collection and Buccal Swab Processing

Saliva was obtained from adult cats using an RNase free technique with sterile gloves. Cats were gently wrapped in blankets, containing their bodies while exposing their heads. A sterile buccal swab was inserted into the cat's mouth and gently brushed against each of the cat's four cheek pouches in a circular motion. The buccal swab was then inserted under the tongue, contacting all salivary glands possible. The buccal swab was then inserted into an RNase free 1.7 mL microcentrifuge tube and cut at the base of the cotton tip with scissors sterilized with ethanol. The tube containing the swab was then labeled. Studies show that saliva has natural stabilizing enzymes that keep mRNA intact for up to three months at room temperature, but samples were refrigerated to inhibit the growth of bacteria until transported to the lab (Wong, et al, Clinical Chemistry, 57(9):1295-302 (2011)). In order to acquire a large enough sample, each cat was swabbed two times to obtain a sufficient starting volume of saliva. The swabbing process is the extent of cat participation in the methods of the invention, and the process itself took under a minute. The swabs were centrifuged at 8,000×g for one minute and then carefully inverted with two sets of sterile tweezers and replaced in the microcentrifuge tubes. The tweezers were sterilized with 70% ethanol between inversions among samples of different test subjects. Once the swabs were inverted, the samples were centrifuged again at 8,000×g for an additional minute to pull remaining saliva out of the cotton swab. The swabs were then discarded, and the supernatant in the microcentrifuge tube was centrifuged briefly. Anywhere from 30 μl to 200 μl of feline whole saliva was collected depending on the cat's mouth size and varying production of saliva.

### Example 2

### Reverse Transcription

Primers were created using NCBI and IDT (Integrated DNA Technologies) online databases and services. The following primers were used:

TABLE 1

| | Forward/ | | Product |
|---|---|---|---|
| Gene | reverse | Sequence | size |
| GAPDH | forward | GCCATCAATGACCCCTTCAT | 82 |
| GAPDH | reverse | GCCGTGGAATTTGCCGT | 82 |
| GAPDH | forward | GGCGTGAACCACGAGAAGTA | 144 |
| GAPDH | reverse | GATGGCATGGACTGTGGTCA | 144 |
| RPS7 | forward | GTCCCAGAAGCCGCACTTT | 74 |
| RPS7 | reverse | CACAATCTCGCTCGGGAAAA | 74 |
| RPS7 | forward | GACGAGTTCGAGTCTGGACAT | 90 |
| RPS7 | reverse | CGTGATGTTCAGCTCCCTCA | 90 |
| Fel d 1-1 | forward | AAGGATGTTAGACGCAGCCC | 89 |
| Fel d 1-1 | reverse | CAACATCCCTCTTCACGGCT | 89 |
| Fel d 1-2 | forward | TTGCTACGTGGAGAACGGAC | 72 |
| Fel d 1-2 | reverse | TTGCTGGAGCTGATGGTTGT | 72 |

Immediately after the pooling of samples, preparations for reverse transcription were made. The resulting cDNA was then used as a template for qPCR. Throughout the procedure, RNase free techniques were used. A lab coat, goggles, and gloves were worn for the duration of the experiment and RNase-Zap was utilized to clean surfaces, pipets, and gloves throughout experimentation.

To prepare for reverse transcription, 10 mM dNTP, EDTA, 10× buffer, 5× buffer, and 0.1 M DTT (all from Qiagen) were taken out of the −20° C. freezer and set on the bench to thaw. When the reagents were fully thawed, each was vortexed, centrifuged, and put on ice. The primers for the genes of interest (Fel d 1-2, Feline GAPDH, and/or Feline RPS7) were also taken from the freezer and set to thaw. The enzymes RNAse OUT™ (Life Technologies, Inc.), SUPERSCRIPT III® (SSIII) (Life Technologies, Inc.), and DNase OUT™ (Life Techologies, Inc.) were left in the freezer until needed.

200 μl RNase-free tubes were clearly labeled with the RNA sample ID (the cat), the target gene, negative or positive, and the date. The negative reverse transcription control tubes did not receive the SSIII. A negative and positive control reaction was run for each gene for each sample. Two genes were tested, so each cat required a Feline GAPDH or RPS7 negative and positive reaction, as well as a Fel d 1-2 negative and positive reaction.

When the primers were fully thawed, each was vortexed and centrifuged three times. Then 5 μl forward and 5 μl reverse primers were added into a 1.7 mL microcentrifuge tube. The primer stock was 100 mM concentration, and the inner primer solution used for reverse transcription was 50 mM. If more inner primer solution was needed for a larger sample size, 5 μl more of both forward and reverse primers were added in equal parts until desired volume was obtained. The primer stock was then returned to the freezer and the inner primers were vortexed vigorously and centrifuged three times. The primers were then set on ice.

The saliva samples were vortexed and centrifuged at >4,000×g three times. 12.6 μl RNase free water was then added to each 200 μl microcentrifuge tube, then 4 μl of sample was added to the microcentrifuge tubes. A total starting volume of 16.6 μl was achieved for each reaction tube. The reactions were then vortexed and centrifuged at >4,000×g three times.

To each reaction, 1 μl RNAse OUT™ (Life Technologies, Inc), 1.6 μl 10× DNase buffer, and 5 μl DNase OUT™ (Life Techologies, Inc.) were added. The DNase OUT™ was added last, and the reactions were quickly pipetted up and down to mix the reaction. A timer was then set for six minutes and the reactions were incubated at room temperature. During incubation, the thermocycler was turned on and the program “70 Hold” was selected and set for a volume of 25 μl. Immediately after six minutes, 1.2 μl 25 mM EDTA was added to each reaction (to inhibit the DNase digestion) and the tube was then flicked to mix. All reactions were then centrifuged >4,000×g.

The samples were then incubated in the thermocycler at 70° C. for five minutes. After incubation the reactions were set on ice for a few minutes to cool and then centrifuged at >4,000×g to collect any condensation. The thermocylcer was then reset to “70 Hold” once more. 2 μl of inner primer was then added to each sample. Feline GAPDH or RPS7 primer was added to its specified tubes and Fel d 1-2 was added to its corresponding tubes. 1 μl 10 mM dNTP was then added to each reaction, and tubes were then vortexed and centrifuged at >4,000×g. Samples were incubated once again in the thermocycler for five minutes. Reactions were put on ice for several minutes, and then centrifuged once more.

While the reactions incubated in the thermoclycler, a mix was made in a 1.7 mL microcentrifuge tube. The number of reactions (plus one extra for pipet error) were multiplied by 6 μl 5× RT buffer, 1 μl RNase Out, and 1 μl 0.1 M DTT. The mix was then vortexed and centrifuged three times. 8 μl of the mix was then added to each reaction. Next 1 μl SSIII was added to the positive reactions only. 1 μl of RNase free water was added to negative reactions only to make up for the volume difference. The samples were then vortexed and centrifuged at >4,000×g and incubated in the thermocycler under the program “RT 50” (volume set for 40 μl). The reactions then incubated for about an hour. In the program “RT 50” incubation temperatures are as follows: 50° C. for 50 minutes, 85° C. for 5 minutes, and 4° C. until samples were removed for further experimentation. The 4° C. mode acts as a freezer to preserve the samples. Samples were then stored in the −20° C. freezer.

The unique aspect of the modified reverse transcription developed in this invention is the low sample volume required for experimentation. Only 4 μL of feline saliva is needed for analysis, whereas ELISA technology requires hundreds of microliters if not milliliters of starting sample.

### Example 3

### qPCR

qPCR is the process where cDNA is amplified within the qPCR thermocycler and specific gene expression is quantified using SYBR Green. cDNA was taken from the −20° C. freezer and set on the bench top to thaw. Diluted primer mixes (10 μl forward primer, 10 μl reverse primer, 80 μl RNase free water, all vortexed and centrifuged three times) were also set on the bench top to thaw. 200 μl microcentrifuge tubes were labeled with the test subject, gene of interest, negative or positive, and the date. Two 1.7 mL tubes were labeled as “Fel d 1 mix” and “GAPDH mix” or “RPS7 mix”.

Once the cDNA completely thawed, all samples were centrifuged at >4,000×g and vortexed three times. 4 μl of cDNA was then pipetted into corresponding tubes according to labels. 36 μl of RNase free water was then added to each sample. cDNA was returned to the freezer and the samples were centrifuged at >4,000×g and vortexed three times once again.

The qPCR primer mixes were then made in the following manner: 5 μl SYBR Green, 0.5 μl primer, and 1.5 μl Rnase free water. A primer mix was created for each gene of interest multiplying the ingredients by the number of reactions plus one extra. Once primer mixes were made, each was centrifuged at >4,000×g and vortexed three times. The diluted primers were then returned to the freezer.

Each sample was tested in triplicate. To begin, 3 μl of RNase free water was pipetted into all water reactions. Each gene had two water reactions treated as a control to monitor erroneous amplification of primer dimers. Next 3 μl of the corresponding cDNA was pipetted into the well plate. Then 7 μl of the corresponding primer mix (Fel d 1 primer for Fel d 1 reactions and the appropriate housekeeping gene) was pipetted into all wells including water reactions.

The plate was then sealed and set in the qPCR thermocycler. qPCR analysis was run for two hours using SYBR Green as a fluorescent dye indicator. The entire qPCR experiment took approximately two hours, at which point cycle times and melt temperatures were recorded and graphs were created (data not shown). In addition, a melt curve was attached to the end of the amplification cycles to indicate product similarities. The cycle times and melt temperatures were recorded and ΔΔCT calculations are done (data not shown). Amplification plots and melt curve graphs were stored on a flash drive for further use.

### Example 4

### Data Analysis

ΔΔCT calculations were used to analyze all data. As all experiments were run in triplicate and therefore produced three CT values, the first step was to average and find the standard deviation for both the positive and negative CT values. This is done for both Fel d 1 (target gene) and GAPDH/RPS7 (the endogenous control/housekeeping gene). Negative controls should always have higher CT values than positive CT samples as larger CT values correlate to lower levels of expression. A table was created recording CT values along with averages and standard deviation values (data not shown). This table was then referred to throughout calculations.

The next step was to normalize the amount of mRNA in the reactions by subtracting the average CT value for GAPDH or RPS7 from the average CT value of Fel d 1 (1). The standard deviation was calculated by finding the square root of the standard deviations for Fel d 1 squared plus GAPDH or RPS7 squared (2). This was done for each cat saliva sample tested. This calculation is called the ΔCT calculation as it compares or normalizes the expression of the target gene to the endogenous control gene in preparation to compare multiple cats side by side (see Livak and Schmittgen, (2001), available from doi:10.1000/meth/2001.1262).

ΔC_{T}= C̅_̅{̅T̅}̅(Fel d 1)− C̅_̅{̅T̅}̅(GAPDH) (1)

\sqrt{s²Fel d 1+s²GAPDH} (2)

To compare the expression of Fel d 1 amongst multiple cats, a ΔΔCT equation was used (3). This was accomplished by subtracting the “calibrator” cat's ΔCT value from the other cat's ΔCT value. The calibrator was always the hypoallergenic cat test subject. The standard deviation value used in ΔΔCT was the same value used for each ΔCT value.

ΔΔC_{T}=ΔC_{T(cat)}−C_{T(calibrator)} (3)

The last mathematical step was calculating the relative quantity (RQ) value (4). This calculation shows the fold difference of expression of Fel d 1 among multiple cats. A maximum and minimum RQ value was also calculated to express the error which is not proportionate due to the exponential nature of CT values.

RQ=2^{-ΔΔC}^{_{T}} (4)

Melt curves displayed a graphical representation of the melting temperature of the product of the qPCR reaction (data not shown). If the triplicates are aligned, only one product is being amplified. If multiple peaks appear on the graph, more than one product is being amplified or primer dimers are present (primers binding to themselves) and the qPCR experiment for that gene is deemed unsuccessful.

A 1.5% gel was run with a ladder and the product of qPCR reactions to see if the product size of the primer matched its intended target. The Fel d 1-2 primer used was supposed to have a product length of 72 bp, and by running a gel with a ladder, this product length was confirmed (data not shown).

### Example 5

### Results

Fel d 1, GAPDH, and RPS7 were all successfully detected and quantified in feline saliva. In one example, the amplification curve obtained represented the expression of Fel d 1 and GAPDH. On the x-axis was the cycle time (CT). Larger CTs indicate lower expression levels and smaller CTs indicate higher expression levels. On the y-axis was a measurement of fluorescence. In the case of cat subject A, there was more Fel d 1 relative to GAPDH. Each reaction was run in triplicates, with a tight amplification curve indicating a more precise experiment. ΔΔCT calculations were processed using the raw data represented in the amplification curves and allowed creation of relative comparisons among cats. As an alternative in some experiments, RPS7 was used as an alternative to GAPDH as the housekeeping gene.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for determining levels of Fel d 1 proteins in a saliva sample obtained from a cat (Felis catus), comprising the steps of:
obtaining the saliva sample from the cat; and
quantifying the mRNA coding for Fel d 1 in the saliva sample.

2. The method of claim 1, further comprising after the obtaining step and before the quantifying step isolating the mRNA from the saliva sample.

3. The method of claim 1, further comprising quantifying mRNA coding for at least one housekeeping gene.

4. The method of claim 3, wherein the housekeeping gene is one or both of glyceraldehyde 3-phosphate dehydrogenase (GAPDH) or ribosomal protein S7 (RPS7).

5. The method of claim 3, further comprising after the obtaining step and before the quantifying step reverse transcribing the mRNA coding for Fel d 1 and the at least one housekeeping gene to produce cDNAs.

6. The method of claim 5, wherein the primers for Fel d 1 comprise at least one of the following pairs of primers: AAGGATGTTAGACGCAGCCC [SEQ ID No. 1] and CAACATCCCTCTTCACGGCT [SEQ ID No. 2] or TTGCTACGTGGAGAACGGAC [SEQ ID No. 3] and TTGCTGGAGCTGATGGTTGT [SEQ ID No. 4].

7. The method of claim 5, wherein the cDNAs are quantified by performing qPCR or dPCR on the cDNA products.

8. The method of claim 5, wherein the cDNAs are quantified by performing massively parallel sequencing on the cDNA products.

9. The method of claim 1, wherein the amount of sample used per reaction is less than 10 μl.

10. The method of claim 9, wherein the amount of sample used per reaction is less than 4 μl.

11. A method for determining levels of Fel d 1 proteins in a saliva or sebaceous gland sample obtained from a cat (Felis catus), comprising the steps of:
obtaining the saliva or sebaceous gland sample from the cat;
reverse transcribing the mRNA coding for Fel d 1 and at least one housekeeping gene from the saliva or sebaceous gland sample; and
performing qPCR, dPCR or massively parallel sequencing on the cDNA products; and
quantifying the mRNA coding for Fel d 1 in the sample.

12. The method of claim 11, wherein the housekeeping gene is one or both of glyceraldehyde 3-phosphate dehydrogenase (GAPDH) or ribosomal protein S7 (RPS7).

13. The method of claim 11, wherein the amount of sample used per reaction is less than 10 μl.

14. The method of claim 13, wherein the amount of sample used per reaction is less than 4 μl.


SEQUENCE LISTING (12 sequences)

SEQ ID NO: 1 - DNA, 20 bp, Artificial Sequence
  PCR primer for Fel d1
aaggatgttagacgcagccc

SEQ ID NO: 2 - DNA, 20 bp, Artificial Sequence
  Sequencing primer for Fel d1
caacatccctcttcacggct

SEQ ID NO: 3 - DNA, 20 bp, Artificial Sequence
  PCR primer for Fel d1.
ttgctacgtggagaacggac

SEQ ID NO: 4 - DNA, 20 bp, Artificial Sequence
  Sequencing primer for Fel d1.
ttgctggagctgatggttgt

SEQ ID NO: 5 - DNA, 20 bp, Artificial Sequence
  PCR primer for GAPDH
gccatcaatgaccccttcat

SEQ ID NO: 6 - DNA, 17 bp, Artificial Sequence
  PCR primer for GAPDH
gccgtggaatttgccgt

SEQ ID NO: 7 - DNA, 20 bp, Artificial Sequence
  PCR primer for GAPDH
ggcgtgaaccacgagaagta

SEQ ID NO: 8 - DNA, 20 bp, Artificial Sequence
  PCR primer for GAPDH
gatggcatggactgtggtca

SEQ ID NO: 9 - DNA, 19 bp, Artificial Sequence
  PCR primer for RPS7
gtcccagaagccgcacttt

SEQ ID NO: 10 - DNA, 20 bp, Artificial Sequence
  PCR primer for RPS7
cacaatctcgctcgggaaaa

SEQ ID NO: 11 - DNA, 21 bp, Artificial Sequence
  PCR primer for RPS7
gacgagttcgagtctggacat

SEQ ID NO: 12 - DNA, 20 bp, Artificial Sequence
  PCR primer for RPS7
cgtgatgttcagctccctca